(19) United States
(12) Patent Application Publication
(10) Pub. No.: US 2017/0112749 A1
(43) Pub. Date: Apr. 27, 2017

(54) ANTIPERSPIRANT COSMETICS COMPRISING SPECIFIC PROTEINS FROM LEGUMES OF THE SPECIES SOYBEAN AND INCLUDING NO ALUMINUM AND/OR ZIRCONIUM HALIDES AND/OR HYDROXY HALIDES

(71) Applicant: Henkel AG & Co. KGaA, Duesseldorf (DE)

(72) Inventors: Bernhard Banowski, Duesseldorf (DE); Stefan Evers, Haan (DE)

(73) Assignee: Henkel AG & Co. KGaA, Duesseldorf (DE)

(21) Appl. No.: 15/399,617

(22) Filed: Jan. 5, 2017

Related U.S. Application Data
(63) Continuation of application No. PCT/EP2015/064411, filed on Jun. 25, 2015.

(30) Foreign Application Priority Data
Jul. 8, 2014 (DE) 10 2014 213 225.8

(51) Int. Cl. A61K 8/64; A61Q 15/00

(52) U.S. Cl. CPC A61K 8/645; A61Q 15/00; A61K 2800/30; A61K 2800/88

(57) ABSTRACT

The invention relates to an antiperspirant cosmetic including at least one specific protein from legumes of the genus Glycine and including no aluminum and/or zirconium halides and/or hydroxy halides. The invention further relates to the use of a specific protein and to a non-therapeutic method for reducing body perspiration. Adding or using said at least one specific protein ensures that the sweat gland(s) is/are effectively influenced, thus resulting in a significant reduction in axillary hyperhidrosis even in the absence of antiperspirant aluminum salts.

### FIELD OF THE INVENTION

The present invention generally relates to an antiperspirant cosmetic without aluminum and/or zirconium halides and/or hydroxy halides, which includes at least one substance selected from the group consisting of cosmetic oils which are liquid at 20° C. and 1013 hPa, odorants and waxes, optionally at least one propellant, and specific proteins from legumes of the genus Glycine. Adding the at least one specific protein results in an influencing of the sweat gland(s).

The present invention further relates to a packaging unit (kit-of-parts) including a cosmetic according to the invention and a cosmetic including at least one antiperspirant active substance.

The present invention additionally relates to the use of specific proteins from legumes of the genus Glycine for at least partially influencing the sweat gland(s).

The present invention also relates to the use of a combination which includes at least one substance selected from the group consisting of cosmetic oils which are liquid at 20° C. and 1013 hPa, odorants and waxes, optionally at least one propellant, and also specific proteins from legumes of the genus Glycine, to reduce and/or prevent perspiration, in particular underarm perspiration or perspiration from other body regions. The combination according to the invention includes no aluminum and/or zirconium halides and/or hydroxy halides.

The present invention further relates to an antiperspirant cosmetic without aluminum and/or zirconium halides and/or hydroxy halides, which includes at least one substance selected from the group consisting of cosmetic oils which are liquid at 20° C. and 1013 hPa, odorants and waxes, optionally at least one propellant, and also at least one specific protein. Adding the at least one specific protein results in an influencing of the sweat gland(s).

The present invention finally relates to a non-therapeutic cosmetic method for preventing and/or reducing body perspiration, in which an antiperspirant cosmetic according to the invention is applied to the skin, in particular to the skin of the underarms, and remains on the skin of the underarms for at least 1 hour, preferably for at least 2 hours, preferably for at least 4 hours, in particular for at least 6 hours.

### BACKGROUND OF THE INVENTION

The washing, cleaning and care of one's body is a basic human need, and modern industry is constantly attempting to meet these human needs in many ways. The lasting elimination or at least reduction in body odor and underarm wetness is particularly important for daily hygiene. In the prior art, numerous specific deodorizing or antiperspirant body care compositions are known which have been developed for use in body regions having a high density of sweat glands, in particular in the underarm area. These are packaged in various administration forms, for example as a powder, in stick form, as an aerosol spray, pump spray, liquid and gel-like roll-on application, cream, gel and as impregnated flexible substrates (deodorant wipes).

Cosmetic antiperspirants of the prior art include, in addition to at least one oil or one wax and an odorant component or a perfume, at least one antiperspirant compound, in particular in the form of aluminum and/or zirconium halides and/or hydroxy halides. On the one hand, these antiperspirant compounds reduce the body's sweat secretion by temporarily narrowing and/or blocking the efferent ducts of the sweat glands, so that the amount of sweat can be reduced by around 20 to 60 percent. On the other hand, due to their antimicrobial action, they have an additional deodorizing effect.

Aluminum and/or zirconium halides and/or hydroxy halides may, in conjunction with the acidic pH of these antiperspirants, lead to unpleasant skin reactions on some users. In addition, the use of the aforementioned antiperspirant compounds may cause stains on the clothing.

There is therefore a need to replace antiperspirant aluminum and/or zirconium halides and/or hydroxy halides with other antiperspirant cosmetic active substances. These antiperspirant active substances should have a good antiperspirant effect, good skin compatibility and should also be easy to formulate. In addition, these antiperspirant active substances should have no negative effect on the storage stability of the antiperspirant cosmetics.

The object of the present invention was to provide an antiperspirant cosmetic which avoids or at least alleviates the disadvantages of the prior art and which has good skin compatibility while at the same time reliably reducing underarm wetness. The antiperspirant cosmetic should also have a high degree of storage stability.

It has now surprisingly been found that the use of at least one protein from legumes of the genus Glycine which brings about a change in light absorption of 1 to 100% in the event of a pH change of at least 0.5 in a pH range from pH 4.0 to pH 8.0, in cosmetics including no antiperspirant aluminum and/or zirconium halides and/or hydroxy halides, results in an antiperspirant effect which is almost comparable to the antiperspirant effect of formulations including aluminum salts and/or aluminum-zirconium complexes.

Furthermore, other desirable features and characteristics of the present invention will become apparent from the subsequent detailed description of the invention and the appended claims, taken in conjunction with this background of the invention.

### BRIEF SUMMARY OF THE INVENTION

An antiperspirant cosmetic including at least one substance selected from the group consisting of cosmetic oils which are liquid at 20° C. and 1013 hPa, odorants and waxes; propellants in a total amount of 0 to 99% by weight, based on the total weight of the antiperspirant cosmetic; and at least one protein in a total amount of 0.1 to 70% by weight, based on the total weight of the antiperspirant cosmetic, wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption, wherein the antiperspirant cosmetic includes no aluminum and/or zirconium halides and/or hydroxy halides.

A packaging unit (kit-of-parts) comprising, packaged separately from one another, at least one first container (C1), including a cosmetic (M1) comprising at least one antiperspirant active substance; and at least one second container (C2), including a cosmetic (M2) comprising at least one protein, wherein the at least one protein occurs in legumes of the genus Glycine, wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption, and wherein the cosmetic (M2) includes no aluminum and/or zirconium halides and/or hydroxy halides.

The use of at least one protein for at least partially influencing the sweat gland(s), wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption.

The use of a combination including at least one substance selected from the group consisting of cosmetic oils which are liquid at 20° C. and 1013 hPa, odorants and waxes; propellants in a total amount of 0 to 99% by weight, based on the total weight of the antiperspirant cosmetic; and at least one protein in a total amount of 0.1 to 70% by weight, based on the total weight of the antiperspirant cosmetic, wherein the at least one protein occurs in legumes of the genus Glycine, wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption, and wherein the combination includes no aluminum and/or zirconium halides and/or hydroxy halides, to reduce and/or prevent perspiration, in particular underarm perspiration or perspiration from other body regions.

An antiperspirant cosmetic including at least one substance selected from the group consisting of cosmetic oils which are liquid at 20° C. and 1013 hPa, odorants and waxes; propellants in a total amount of 0 to 99% by weight, based on the total weight of the antiperspirant cosmetic; and at least one soybean protein in a total amount of 0.1 to 70% by weight, based on the total weight of the antiperspirant cosmetic, wherein the soybean protein is hydrolyzed and/or wherein the soybean protein includes a lauryldimoniumhydroxypropyl group, wherein the antiperspirant cosmetic includes no aluminum and/or zirconium halides and/or hydroxy halides.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is an antiperspirant cosmetic including
a) at least one substance selected from the group consisting of cosmetic oils which are liquid at 20° C. and 1013 hPa, odorants and waxes,
b) propellants in a total amount of 0 to 99% by weight, based on the total weight of the antiperspirant cosmetic, and
c) at least one protein in a total amount of 0.1 to 70% by weight, based on the total weight of the antiperspirant cosmetic, wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption,
   wherein the antiperspirant cosmetic includes no aluminum and/or zirconium halides and/or hydroxy halides.

Without wishing to be limited to this theory, the use of the at least one protein from legumes of the genus Glycine with the aforementioned specific physical properties in the antiperspirant cosmetics according to the invention leads to a targeted influencing of the sweat gland(s). This targeted influencing of the sweat gland(s) may consist for example in a gel formation of the at least one protein at pH values which prevail exclusively within the efferent ducts of the sweat glands. In this way, effective blocking of the efferent ducts of the sweat glands can be ensured without the antiperspirant effect of the cosmetic according to the invention being reduced due to premature undesired gel formation as a result of adding the at least one specific protein. However, the targeted the influencing of the sweat gland(s) may also consist in a disruption of the charge equilibrium within the sweat gland(s), which leads to an influencing of sweat production, in particular to a reduction in sweat production. An effective reduction in underarm sweat is thus ensured even in the absence of antiperspirant aluminum and/or zirconium halides and/or hydroxy halides.

According to the invention, the term “antiperspirant” will be understood to mean a decrease or reduction in perspiration of the sweat glands of the body.

In addition, within the context of the present invention, the term “aluminum and/or zirconium halides and/or hydroxy halides” will be understood to mean in particular aluminum and zirconium chlorides, bromides and iodides as well as compounds of formulae Al(OH)_{y}X and Zr(OH)_{z}X, where X in the aforementioned formulae represents a halide ion.

Furthermore, within the context of the present invention, the term “cosmetic oil” will be understood to mean an oil which is suitable for cosmetic use and which is not miscible with water in all quantities. The cosmetic oil used according to the invention is neither an odorant nor an essential oil.

Moreover, within the context of the present invention, the term “odorants” will be understood to mean substances having a molar mass of 74 to 300 g/mol which include at least one osmophoric group in the molecule and which have an odor and/or taste, that is to say they are capable of stimulating the receptors of the hair cells of the olfactory system. Osmophoric groups are groups covalently bonded to the molecule backbone, in the form of hydroxyl groups, formyl groups, oxo groups, alkoxycarbonyl groups, nitrile groups, nitro groups, azide groups, etc. In this connection, perfume oils which are liquid at 20° C. and 1013 hPa, perfumes or perfume oil constituents also fall under the term “odorants” within the context of the present invention.

In addition, within the context of the present invention, the term “waxes” will be understood to mean substances which at 20° C. are kneadable or solid to brittle and hard, have a coarse to fine crystalline structure and are colored and translucent to opaque, but not vitreous. Furthermore, these substances melt above 25° C. without decomposing, are slightly liquid (less viscous) just above the melting point, have a greatly temperature-dependent consistency and solubility, and can be polished under slight pressure.

According to the invention, the term “protein” denotes chemical compounds which form amino acid condensation products which are linked by peptide bonds in an acid amide-like manner. The number of amino acids in the proteins is preferably at least 2 and at most 1000 amino acids. According to the invention, the term “protein” will also be understood to mean hydrolysates of a protein, which include protein fractions with different amino acid sequences and molecular weights. Within the context of the present invention, this term will also be understood to mean mixtures of proteins occurring in legumes of the genus Glycine.

Furthermore, within the context of the present invention, the term “legumes” will be understood to mean plants which form a legume from just one carpel, the legume splitting open along a front and back seam as it ripens. According to the invention, however, this term will also be understood to mean the seeds, in particular the ripe seeds, of the aforementioned plants.

In addition, the expression “change in the light absorption of the at least one protein” will be understood to mean both the positive and negative change in the light permeability of the sample mixture, in particular the protein solution, and also the absorption of light by the at least one protein or the sample mixture.

Furthermore, the expression “pH change” will be understood to mean the continuous changing of the pH. The continuous changing of the pH can be achieved for example by titration, or uniform addition, of a base or acid.

According to the invention, the term “sample mixture” denotes a mixture of the at least one specific protein with a solvent, in particular water, buffer or salt-including aqueous solutions.

Moreover, the term “fatty acids”, as used in the context of the present invention, will be understood to mean aliphatic carboxylic acids which have unbranched or branched carbon radicals having 4 to 40 carbon atoms. The fatty acids used in the context of the present invention may be both naturally occurring and synthetically produced fatty acids. Furthermore, the fatty acids may be monounsaturated or polyunsaturated.

Finally, within the context of the present invention, the term “fatty alcohols” will be understood to mean aliphatic, monovalent, primary alcohols which have unbranched or branched hydrocarbon radicals having 4 to 40 carbon atoms. The fatty alcohols used in the context of the invention may also be monounsaturated or polyunsaturated.

In the present case, unless indicated otherwise, the specification % by weight relates to the total weight of the antiperspirant cosmetic according to the invention.

As a first constituent a), the cosmetics according to the invention include at least one substance selected from the group consisting of cosmetic oils which are liquid at 20° C. and 1013 hPa, odorants and waxes.

Within the context of the present invention, the cosmetic oil which is liquid at 20° C. and 1013 hPa is selected from the group consisting of (i) volatile cyclic silicone oils, in particular cyclic and linear silicone oils; (ii) volatile non-silicone oils, in particular liquid paraffin oils and isoparaffin oils; (iii) non-volatile silicone oils; (iv) non-volatile non-silicone oils; and (v) mixtures thereof.

According to the invention, the term “volatile oil” denotes oils which, at 20° C. and at an ambient pressure of 1013 hPa, have a vapor pressure of 2.66 Pa to 40,000 Pa (0.02 to 300 mm Hg), preferably 10 to 12,000 Pa (0.1 to 90 mm Hg), more preferably 13 to 3000 Pa (0.1 to 23 mm Hg), in particular 15 to 500 Pa (0.1 to 4 mm Hg).

Moreover, within the context of the present invention, the term “non-volatile oils” will be understood to mean oils which, at 20° C. and at an ambient pressure of 1013 hPa, have a vapor pressure of less than 2.66 Pa (0.02 mm Hg).

It may be preferred according to the invention to use mixtures of volatile silicone oils and volatile non-silicone oils in the antiperspirant cosmetics according to the invention, since a drier skin feel is achieved as a result. Furthermore, it may be preferred within the context of the present invention if the antiperspirant cosmetics include a non-volatile silicone oil and/or a non-volatile non-silicone oil in order to mask insoluble constituents such as talc or ingredients that have dried onto the skin.

According to the invention, particular preference is given to using mixtures of non-volatile and volatile cosmetic oils, since in this way parameters such as skin feel, residue visibility and stability of the antiperspirant cosmetic according to the invention can be adjusted and thus can be better adapted to the requirements of consumers.

According to one preferred embodiment of the present invention, the cosmetic oil which is liquid at 20° C. and 1013 hPa is included in a total amount of 0.02 to 98% by weight, preferably 2 to 85% by weight, preferably 4 to 75% by weight, more preferably 6 to 70% by weight, yet more preferably 8 to 60% by weight, in particular 8 to 20% by weight, based on the total weight of the antiperspirant cosmetic.

At least one odorant may also be included as constituent a) of the cosmetics according to the invention. However, use will preferably be made of mixtures of different odorants which together produce a pleasant fragrance.

Particularly pleasant-smelling antiperspirant cosmetics according to the invention are obtained if the at least one odorant is included in a total amount of 0.00001 to 15% by weight, preferably 0.001 to 9% by weight, preferably 0.01 to 8% by weight, more preferably 0.1 to 7% by weight, yet more preferably 0.2 to 6% by weight, in particular 0.2 to 2% by weight, based on the total weight of the antiperspirant cosmetic.

Furthermore, the antiperspirant cosmetics according to the invention may include a wax as constituent a). This wax is preferably selected from the group consisting of (i) fatty acid glycerol mono-, di- and triesters; (ii) Butyrospermum parkii (shea butter); (iii) esters of saturated, monovalent C₈₋₁₈ is alcohols with saturated C₁₂₋₁₈ monocarboxylic acids; (iv) linear, primary C₁₂-C₂₄ alkanols; (v) esters of a saturated, monovalent C₁₆₋₆₀ alkanol and a saturated C₈-C₃₆ monocarboxylic acid; (vi) glycerol triesters of saturated linear C₁₂₋₃₀ carboxylic acids, which may be hydroxylated; (vii) natural plant waxes; (viii) animal waxes; (ix) synthetic waxes; and (x) mixtures thereof.

Within the context of the present invention, it is preferred if the wax is included in a total amount of 0.01 to 50% by weight, preferably 3 to 40% by weight, preferably 5 to 30% by weight, in particular 6 to 25% by weight, based on the total weight of the antiperspirant cosmetic.

According to one embodiment of the present invention, it may be provided that the antiperspirant cosmetics according to the invention include, as constituent b), a propellant in a total amount of 0 to 99% by weight, based on the total weight of the antiperspirant cosmetic. If the cosmetics according to the invention include a propellant, the latter is preferably included in a total amount of 1 to 98% by weight, preferably 20 to 90% by weight, preferably 30 to 85% by weight, in particular 40 to 75% by weight, based on the total weight of the antiperspirant cosmetic. In this case, the cosmetics according to the invention are packaged as aerosols driven by a propellant gas. Preferred propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, and tetrafluoropropene, namely either individually or in mixtures thereof. Hydrophilic propellant gases, such as for example carbon dioxide, can also advantageously be used within the context of the present invention if the proportion of hydrophilic gases is selected to be low and lipophilic propellant gas (for example propane/butane) is present in excess. Particular preference is given to propane, n-butane, isobutane and mixtures of these propellant gases. It has been found that the use of n-butane as the sole propellant gas may be particularly preferred according to the invention.

As the third constituent c), the antiperspirant cosmetic according to the invention includes at least one specific protein from legumes of the genus Glycine. The genus bearing the Latin name Glycine designates legumes in the form of soybeans.

Within the context of the present invention, a particularly effective reduction in underarm sweat by virtue of the at least one specific protein is achieved if the at least one protein is included in a total amount of 0.5 to 60% by weight, preferably 1.0 to 50% by weight, preferably 1.5 to 40% by weight, more preferably 2.0 to 30% by weight, in particular 2.0 to 20% by weight, based on the total weight of the antiperspirant cosmetic. Without wishing to be limited to this theory, the use of the aforementioned amounts of the at least one specific protein results in a significant influencing of the sweat gland(s) due to gel formation of the protein in the efferent ducts of the sweat glands or due to influencing of the charge equilibrium within the sweat gland(s). In this way, an excellent antiperspirant effect is ensured. Furthermore, the use of the aforementioned amounts of the at least one specific protein does not lead to unstable formulations, so that the stability of the antiperspirant cosmetics according to the invention is ensured even over long storage times.

Particularly good results with regard to decreasing and/or reducing underarm wetness and with regard to skin compatibility and storage stability are obtained if the at least one protein has an average molecular weight M_{w} of 150 to 100,000 Da, preferably 180 to 50,000 Da, preferably 200 to 10,000 Da, more preferably 250 to 8000 Da, in particular 300 to 5000 Da. The average molecular weight M_{w} can be determined for example by means of gel permeation chromatography (GPC).

According to one preferred embodiment of the present invention, the at least one protein has an isoelectric point which lies in the range from pH 4.0 to pH 10.0, preferably from pH 4.0 to pH 9.5, in particular from pH 4.0 to pH 8.0. Within the context of the present invention, proteins which have an isoelectric point in the aforementioned pH range have proven to be particularly advantageous with regard to the antiperspirant effect and the stability of the cosmetics according to the invention.

Within the context of the present invention, a particularly high antiperspirant effect, skin compatibility and storage stability is ensured if the at least one protein brings about a change in light absorption in the event of a pH change of at least 0.5 in a pH range from pH 4.5 to pH 7.5, in particular from pH 5.0 to pH 7.0, at a concentration of 0.001 to 10% by weight protein, based on the total weight of the sample mixture used for the pH measurement, and at a temperature of 20° C. Without wishing to be limited to this theory, the use of the at least one specific protein which brings about a change in light absorption in a particular pH range results in a significantly increased influencing of the sweat gland(s) due to pH-selective gel formation in the efferent ducts of the sweat glands or due to a disruption of the charge equilibrium of the sweat gland(s), so that an excellent antiperspirant effect of the cosmetics according to the invention is ensured, which is comparable to the antiperspirant effect of prior art cosmetics including aluminum salt or aluminum-zirconium salt.

Within the context of the present invention, it is preferred if the pH change is achieved by adding hydrogen carbonates or carbonates, in particular sodium hydrogen carbonates.

According to one preferred embodiment of the present invention, the at least one protein is selected from the group consisting of (i) unmodified proteins; (ii) hydrolyzed proteins; (iii) chemically modified proteins, in particular hydrophobically and/or cationically and/or anionically modified proteins; (iv) physically modified proteins, in particular fractionated and/or purified and/or irradiated proteins; (v) hydrolyzed unmodified proteins; (vi) hydrolyzed and chemically modified proteins, in particular hydrolyzed and hydrophobically and/or cationically and/or anionically modified proteins; (vii) hydrolyzed and physically modified proteins, in particular fractionated and/or purified and/or irradiated proteins; and (viii) mixtures thereof.

According to the invention, the term “unmodified proteins” will be understood to mean proteins which have not been treated either by chemical methods, such as for example hydrolysis or chemical modification, or by physical methods, such as for example purification, separation and irradiation.

Furthermore, according to the invention, the term “hydrolyzed proteins” or “protein hydrolysates” will be understood to mean proteins which are produced by chemical hydrolysis, in particular alkaline or acid hydrolysis, by enzymatic hydrolysis and/or by a combination of both types of hydrolysis. All hydrolytically acting enzymes, such as alkaline proteases for example, are suitable for the enzymatic degradation. Within the context of the present invention, mixtures of individual amino acids which are obtained merely by mixing the pure substances of the amino acids, and also total hydrolysates which consist only of individual amino acids, do not fall under the term “hydrolyzed proteins” or “protein hydrolysates”.

In addition, within the context of the present invention, the term “chemically modified proteins” will be understood to mean proteins which are obtained by chemically reacting the reactive groups of the proteins, in particular the hydroxyl, amine, imidazole, guanidino and/or thiol groups of the side chains of the amino acids of the protein, with hydrophobic and/or cationic and/or anionic compounds.

Moreover, within the context of the present invention, the term “physically modified proteins” will be understood to mean proteins which have been modified by a physical action, in particular by heat and/or light and/or fractionation.

Within the context of this embodiment, it is particularly preferred if the at least one protein is selected from the group consisting of chemically modified, in particular hydrophobically modified, proteins. In this connection, the hydrophobically modified protein has one or more C₄₋₃₀ carbon chains, wherein the C₄₋₃₀ hydrocarbon chains may be linear, cyclic, branched, unbranched, saturated, unsaturated and aromatic, and wherein the C₄₋₃₀ hydrocarbon chains are bound to the protein radical via ether and/or ester and/or amine and/or amide bonds.

In addition, within the context of this embodiment, it is preferred if the at least one protein is selected from the group consisting of chemically modified, in particular cationically modified, proteins. The cationically modified protein therefore preferably includes one or more radical(s) of formula R¹—N⁺(CH₃)₂—CH₂—CH(OH)—CH₂—X—R, in which R¹ represents an alkyl group having 1 to 30 carbon atoms, an alkenyl group having 1 to 30 carbon atoms, a hydroxyalkyl group having 1 to 30 carbon atoms, in particular a methyl group, a C₁₀₋₁₄ alkyl or a C₁₀₋₁₄ alkenyl group, X represents O, N or S, and R represents the protein radical. The cationization of the proteins with the above-described radicals can be achieved by reacting the proteins with the appropriate halides of the above formula, wherein the above-described radicals can be bound to the protein via ether and/or ester and/or amide and/or amine bonds. Within the context of the present invention, the term “protein radical” will be understood to mean the backbone of the protein in question, which is formed by the linkage of amino acids and to which the cationic group is bound via the aforementioned bonds.

With regard to the antiperspirant effect, the skin compatibility and the storage stability of the cosmetics according to the invention, it is particularly preferred if the at least one protein is hydrolyzed and/or if the at least one protein includes at least one lauryldimoniumhydroxypropyl group. Without wishing to be limited to this theory, the use of these specific proteins results in a significantly increased influencing of the sweat gland(s) due to pH-selective gel formation or disruption of the charge equilibrium within the efferent ducts of the sweat glands. In this way, an excellent antiperspirant effect of the cosmetics according to the invention is ensured, which is comparable to the antiperspirant effect of prior art cosmetics including aluminum salt or aluminum-zirconium salt. In addition, the use of these specific proteins does not lead to a negative interaction with other ingredients in the antiperspirant cosmetic, so that a high degree of storage stability of the antiperspirant cosmetics according to the invention is ensured. Moreover, the cosmetics according to the invention have a high degree of skin compatibility.

In this connection, it has proven to be advantageous if the at least one protein includes lysine in 1.5 to 6.5 mol %, histidine in 1.0 to 2.5 mol % and arginine in 4.5 to 6.5 mol %, based on the total amount of all amino acids of the protein. The proportion of the aforementioned amino acids can be carried out for example by chromatographic determination using sulfonated polystyrene resins.

It has also proven to be advantageous in this connection if the at least one protein has a calcium content of 10 to 600 mg, a potassium content of 650 to 850 mg, a phosphorus content of 800 to 1400 mg and a sulfur content of 1000 to 1600 mg, in each case based on 1 kg of the protein. The specified contents can be determined for example by means of atom emission spectrometry (ICP-OES) after microwave digestion with nitric acid.

According to another particularly preferred embodiment of the present invention, the at least one protein brings about a change in light absorption of 1.5 to 90%, preferably 2 to 80%, preferably 2.5 to 70%, more preferably 3 to 65%, in particular 3.5 to 60%. Within the context of the present invention, particularly proteins from legumes of the genus Glycine which bring about the aforementioned change in light absorption lead to an excellent antiperspirant effect. The change in light absorption may be based on a change in the light permeability of the sample mixture, in particular due to clouding, and on the absorption of light by the sample mixture, in particular by the protein itself.

The changes in light absorption in the event of a pH change of at least 0.5, on which this invention is based, can be determined by measuring the light transmission of a light beam through the sample mixture. The light transmission is measured using a Methrom Optrode 6.1115.000 at a wavelength of 574 nm (green-yellow) in mV (resolution 0.1 mV) in an open sample vessel at 23° C. and 1013 mbar. The pH change in the pH range from 4.0 to 8.0 is achieved by slowly and continuously adding a carbonate or hydrogen carbonate solution, preferably a 1% strength by weight sodium hydrogen carbonate solution, to the sample mixture while measuring the pH using a pH electrode and while stirring at a speed of 750 to 850 rpm. The change in light absorption brought about by the at least one protein can be calculated according to the formula ΔL=[(|Lᵢ|/|L₀|]*100. In said formula, Lᵢ is the light transmission after a pH change of at least 0.5 in the pH range from 4.0 to 8.0, preferably from pH 4.5 and 7.5, in particular from pH 5.0 and 7.0. In said formula, L₀ is the difference of the light transmission at pH 4.0 and at pH 8.0, preferably at pH 4.5 and at pH 7.5, in particular at pH 5.0 and at pH 7.0, that is to say for example the light transmission at pH 8.0 minus the light transmission at pH 4.0. The at least one specific protein in the antiperspirant cosmetics according to the invention gives rise to a change in light absorption of 1 to 100%, which can be determined by the above method. However, the present invention is not limited to antiperspirant cosmetic compositions which include at least one specific protein that gives rise to a change in light absorption of 1 to 100% as determined by the above method. It also encompasses antiperspirant cosmetic compositions which include at least one specific protein that gives rise to a change in light absorption of 1 to 100% by other methods.

Within the context of the present invention, it is preferred if the concentration of the at least one protein in the mixture used to determine the change in light absorption is from 0.005 to 10% by weight, preferably from 0.05 to 5% by weight, preferably from 0.07 to 3% by weight, in particular from 0.09 to 2% by weight, based on the total weight of the sample mixture used to determine the change in light absorption.

According to the invention, the at least one protein preferably gives rise to a change in light absorption in the event of a pH change of at least 0.5 and at most 3.5, preferably at least 0.5 and at most 2.5, in particular at least 0.5 and at most 1.5. The pH change can be achieved in particular by adding acids or bases, preferably bases in the form of carbonates or hydrogen carbonates, in the appropriate quantity.

According to another preferred embodiment of the present invention, the antiperspirant cosmetic has a pH of pH 2 to pH 10. Within this range, a stable formulation of the cosmetics according to the invention is possible without undesired interactions occurring between the ingredients of the antiperspirant cosmetics according to the invention. According to the invention, the setting of the desired pH may take place by using acids and bases which are known to a person skilled in the art and which are customary in antiperspirant cosmetics.

It is also preferred according to the invention if the antiperspirant cosmetic additionally includes at least one preservative. Preservatives which are preferred according to the invention are formaldehyde releasers iodopropynyl butylcarbamate, parabens, phenoxyethanol, ethanol, benzoic acid and salts thereof, dibromodicyanobutane, 2-bromo-2-nitropropane-1,3-diol, imidazolidinyl urea, 5-chloro-2-methyl-4-isothiazolin-3-one, 2-chloroacetamide, benzalkonium chloride, benzyl alcohol, salicylic acid and salicylates. Other preservatives which can be used in the context of the present invention are the substances mentioned in Annex 6 of the Cosmetics Regulation as well as cosmetic raw materials with preserving properties, or raw materials which support or enhance the preserving effect of the aforementioned preservatives. The preservatives are preferably included in a total amount of 0.01 to 10% by weight, preferably 0.1 to 7% by weight, preferably 0.2 to 5% by weight, in particular 0.3 to 2.0% by weight, based on the total weight of the antiperspirant cosmetic.

Within the context of the present invention, it is preferred if the antiperspirant cosmetic is in the form of a water-in-oil emulsion. This may be in particular a sprayable water-in-oil emulsion, which can be sprayed by means of a propellant. In this connection, it is preferred if the antiperspirant cosmetic according to the invention in the form of a water-in-oil emulsion includes the at least one protein in a total amount of 0.1 to 70% by weight, preferably 0.5 to 60% by weight, preferably 1.0 to 50% by weight, more preferably 1.5 to 40% by weight, yet more preferably 2.0 to 30% by weight, in particular 2.0 to 20% by weight, based on the total weight of the antiperspirant cosmetic.

However, it may equally be preferred according to the invention if the antiperspirant cosmetic is in the form of an oil-in-water emulsion. In this case, the cosmetic according to the invention is preferably sprayed as a propellant-free pump spray or squeeze spray or applied as a roll-on. In this connection, it is preferred if the antiperspirant cosmetic in the form of an oil-in-water emulsion includes the at least one protein in a total amount of 0.1 to 70% by weight, preferably 0.5 to 60% by weight, preferably 1.0 to 50% by weight, more preferably 1.5 to 40% by weight, yet more preferably 2.0 to 30% by weight, in particular 2.0 to 20% by weight, based on the total weight of the antiperspirant cosmetic.

According to another preferred embodiment of the present invention, the cosmetics according to the invention may include only a low content of free water or no free water. Within the context of the present invention, free water will be understood to mean water which is different from water of crystallization, hydration water or similarly molecularly bound water of the constituents used. The antiperspirant cosmetic preferably includes free water in a total amount of less than 10% by weight, preferably less than 8% by weight, preferably less than 5% by weight, more preferably less than 3% by weight, yet more preferably less than 1% by weight, in particular 0% by weight, based on the total weight of the antiperspirant cosmetic.

Within the context of a further embodiment, however, it is likewise preferred according to the invention if the antiperspirant cosmetic is in the form of an aqueous, aqueous-alcoholic or aqueous-glycolic solution. Since the cosmetics according to the invention include no antiperspirant aluminum and/or zirconium halides and/or hydroxy halides which have a reduced antiperspirant effect due to the addition of protic solvents, according to the invention protic solvents such as aqueous solutions can be used to formulate the antiperspirant cosmetics according to the invention, without any significant reduction in the antiperspirant effect occurring. The addition of the at least one specific protein therefore ensures an effective influencing of the sweat gland(s) and thus an excellent antiperspirant effect, even when protic solvents are used.

In connection with this embodiment of the present invention, it has surprisingly been found that the influencing of the sweat gland(s) by the at least one specific protein can be significantly increased if the antiperspirant cosmetics according to the invention include free water in an amount of 5 to 99% by weight, based on the total weight of the antiperspirant cosmetic. In one particularly preferred embodiment of the present invention, therefore, the antiperspirant cosmetic includes free water in a total amount of 5 to 96% by weight, preferably 15 to 80% by weight, preferably 30 to 70% by weight, in particular 40 to 60% by weight, based on the total weight of the antiperspirant cosmetic.

In connection with this embodiment, it is also preferred if the antiperspirant cosmetic includes ethanol in a total amount of 1 to 99% by weight, preferably 5 to 70% by weight, preferably 7 to 50% by weight, in particular 10 to 30% by weight, based on the total weight of the antiperspirant cosmetic. As previously mentioned, by using the at least one specific protein, even large amounts of protic solvents such as ethanol can be used without adversely affecting the antiperspirant effect of the antiperspirant cosmetic according to the invention.

The antiperspirant cosmetic according to the invention may be applied by various methods. According to one preferred embodiment, the antiperspirant cosmetic is packaged as a spray application. The spray application takes place by means of a spray device which includes in a container a filling of the liquid, viscous-flowable, suspension-like or pulverulent antiperspirant cosmetic according to the invention. The filling may be under the pressure of a propellant (pressurized gas cans, pressurized gas packagings, aerosol packagings), or it may be a mechanically operated pump atomizer without a propellant gas (pump sprays/squeeze bottles). The atomization of the antiperspirant cosmetic may take place physically, mechanically or electromechanically, for example by piezo effects or electric pumps.

The antiperspirant cosmetic may also preferably be packaged as a stick, soft solid, cream, gel, roll-on, loose or compact powder. The formulation of the antiperspirant cosmetics according to the invention in a particular administration form, such as for example an antiperspirant roll-on, an antiperspirant stick or antiperspirant gel, preferably depends on the requirements of the intended use. Depending on the intended use, the antiperspirant cosmetics according to the invention may therefore be in solid, semi-solid, liquid, dispersed, emulsified, suspended, gel-like, multiphase or powder form. Within the context of the present invention, the term liquid also encompasses any type of dispersions of solids in liquids. Furthermore, within the context of the present invention, multiphase antiperspirant cosmetics according to the invention will be understood to mean cosmetics which have at least 2 different phases with a phase separation, and in which the phases may be arranged horizontally, that is to say one above the other, or vertically, that is to say next to one another. Application may take place for example using a roller ball applicator or by means of a solid stick.

Within the context of the present invention, it may likewise be preferred if the antiperspirant cosmetic is included on and/or in a disposable substrate, selected from the group consisting of wipes, pads and wads. Particular preference is given to wet wipes, that is to say wet wipes which are pre-prepared for the user and are preferably packaged individually, as are well known for example from the field of glass cleaning or from the field of moist toilet paper. Such wet wipes, which advantageously may also include preservatives, are impregnated with an antiperspirant cosmetic according to the invention or have the latter applied thereto, and are preferably packaged individually. Preferred substrate materials are selected from porous flat wipes. These wipes include wipes made from woven and unwoven (nonwoven) synthetic and natural fibers, felt, paper or foam, such as hydrophilic polyurethane foam. Deodorizing or antiperspirant substrates which are preferred according to the invention may be obtained by soaking or impregnating or even by melting an antiperspirant cosmetic according to the invention onto a substrate.

According to the invention, the antiperspirant cosmetic preferably includes at least one wide auxiliary selected from the group consisting of (i) emulsifiers and/or surfactants; (ii) thickeners; (iii) chelating agents; (iv) deodorant active substances; (v) monovalent and/or polyvalent alcohols and/or polyethylene glycols; (vi) skin-cooling active substances; (vii) pH adjusters; (viii) skin-care active substances, such as moisturizers, skin-soothing substances, skin-brightening substances, skin-smoothing substances; and (ix) mixtures thereof.

Emulsifiers and surfactants which are preferably suitable according to the invention are selected from anionic, cationic, nonionic, amphoteric, in particular ampholytic and zwitterionic emulsifiers and surfactants. Surfactants are amphiphilic (bifunctional) compounds which consist of at least one hydrophobic and at least one hydrophilic moiety. The hydrophobic radical is preferably a hydrocarbon chain having 8 to 28 carbon atoms, which may be saturated or unsaturated, linear or branched. With particular preference, this C₈-C₂₈ alkyl chain is linear.

In order to thicken the antiperspirant cosmetics according to the invention, use is preferably made of substances selected from cellulose ethers, xanthan gum, sclerotium gum, succinoglucans, polygalactomannans, pectins, agar, carrageenan, tragacanth, gum arabic, karaya gum, tara gum, gellan gum, gelatin, propylene glycol alginate, alginic acids and salts thereof, polyvinylpyrrolidones, polyvinyl alcohols, polyacrylamides, physically (for example by pre-gelatinization) and/or chemically modified starches, acrylic acid/acrylate copolymers, acrylic acid/acrylamide copolymers, acrylic acid/vinylpyrrolidone copolymers, acrylic acid/vinylformamide copolymers, and polyacrylates. Particularly preferred thickeners are also selected from carbomers. Carbomers are thickening crosslinked polymers of acrylic acid, methacrylic acid and salts thereof. The crosslinking may take place by means of polyfunctional compounds such as polyalkylene ethers of polysaccharides or polyalcohols, for example sucrose allyl ether, pentaerythritol allyl ether, propylene allyl ether. Within the context of the present invention, preference is given to homopolymers of acrylic acid or salts thereof, which are crosslinked with a pentaerythritol allyl ether, a sucrose allyl ether or a propylene allyl ether. One thickener which can be used in the context of the present invention is a copolymer of C₁₀₋₃₀ alkyl acrylate, acrylic acid, methacrylic acid and esters thereof, which is crosslinked with a sucrose allyl ether or a pentaerythritol allyl ether. Thickeners based on carbomers are the products available under the trade name Carbopol® (BF Goodrich, Ohio, USA), such as for example Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol ultrez 10, Carbopol ultrez 20, or Carbopol ultrez 21.

Lipophilic thickeners can also be used to thicken the antiperspirant cosmetics according to the invention. Lipophilic thickeners which are preferred according to the invention are selected from hydrophobicized clay minerals, bentonites, pyrogenic silicas and derivatives thereof.

In order to further aid the influencing of the sweat gland(s) by the at least one specific protein, it may be advantageous to add to the antiperspirant cosmetics according to the invention at least one chelating agent in a total amount of 0.01 to 3.0% by weight, preferably 0.02 to 1.0% by weight, in particular 0.05 to 0.1% by weight, based on the total weight of the antiperspirant cosmetic according to the invention. Within the context of the present invention, preferred chelating agents are selected from the group consisting of β-alaninediacetic acid, cyclodextrin, diethylenetriaminepentamethylenephosphonic acid, sodium, potassium, calcium disodium, ammonium and triethanolamine salts of ethylenediaminetetraacetic acid (EDTA), etidronic acid, hydroxyethylethylenediaminetetraacetic acid (HEDTA) and sodium salts thereof, sodium salts of nitrilotriacetic acid (NTA), diethylenetriaminepentaacetic acid, phytic acid, hydroxypropylcyclodextrin, methylcyclodextrin, pentasodium aminotrimethylene phosphonate, pentasodium ethylenediaminetetramethylene phosphonate, pentasodium diethylenetriamine pentaacetate, pentasodium triphosphate, potassium EDTMP, sodium EDTMP, sodium dihydroxyethylglycinate, sodium phytate, sodium polydimethylglycinophenol sulfonate, tetrahydroxyethylethylenediamine, tetrahydroxypropylethylenediamine, tetrapotassium etidronate, tetrasodium etidronate, tetrasodium iminodisuccinate, trisodium ethylenediamine disuccinate, tetrasodium-N,N-bis(carboxymethyl)glutamate, tetrasodium-DL-alanine-N,N-diacetate, and desferrioxamine.

The deodorizing effect of the antiperspirant cosmetics according to the invention can be further increased if at least one deodorant active substance with an antibacterial and/or bacteriostatic and/or enzyme-inhibiting and/or odor-neutralizing and/or odor-absorbing effect is included in a total amount of 0.0001 to 40% by weight, preferably 0.2 to 20% by weight, preferably 1 to 15% by weight, in particular 1.5 to 5% by weight, based on the total weight of the antiperspirant cosmetic according to the invention. If ethanol is used in the cosmetics according to the invention, this is not deemed to be a deodorant active substance in the context of the present invention but rather is deemed to be a constituent of the carrier.

Preferred antiperspirant cosmetics according to the invention also include at least one water-soluble polyvalent C₂₋₉ alkanol having 2 to 6 hydroxyl groups and/or at least one water-soluble polyethylene glycol having 3 to 50 ethylene oxide units, and mixtures thereof. The aforementioned deodorant active substances in the form of 1,2-alkanediols do not fall under this definition.

According to another embodiment of the present invention, the antiperspirant cosmetics also include at least one skin-cooling active substance. Skin-cooling active substances which are suitable according to the invention are for example menthol, isopulegol and also menthol derivatives, for example menthyl lactate, menthyl glycolate, menthyl ethyl oxamate, menthylpyrrolidonecarboxylic acid, menthyl methyl ether, menthoxypropanediol, menthone glycerin acetal (9-methyl-6-(1-methylethyl)-1,4-dioxaspiro(4.5)decane-2-methanol), monomenthyl succinate, 2-hydroxymethyl-3,5,5-trimethylcyclohexanol and 5-methyl-2-(1-methylethyl)cyclohexyl-N-ethyl oxamate. Preferred skin-cooling active substances are menthol, isopulegol, menthyl lactate, menthoxypropanediol, menthylpyrrolidonecarboxylic acid and 5-methyl-2-(1-methylethyl)cyclohexyl-N-ethyl oxamate, and also mixtures of these substances, in particular mixtures of menthol and menthyl lactate, menthol, menthol glycolate and menthyl lactate, menthol and menthoxypropanediol or menthol and isopulegol.

As pH adjusters, preferably acids and/or alkalizing agents and/or buffers are used according to the invention. As acids, preferably inorganic acids (such as for example hydrochloric acid, sulfuric acid or phosphoric acid) or organic acids (such as for example citric acid, tartaric acid or malic acid) are used according to the invention. The alkalizing agents which can be used according to the invention are preferably selected from the group formed of ammonia, basic amino acids, alkali hydroxides, carbonates and hydrogen carbonates, alkanolamines, for example amino-2-methyl-1-propanol, monoethanolamine, triethanolamine, diethanolamine and triisopropanolamine, alkali metal metasilicates, urea, morpholine, N-methylglucamine, imidazole, alkali phosphates and alkali hydrogen phosphates. As alkali metal ions, use is preferably made of lithium, sodium, potassium, in particular sodium or potassium. Suitable buffer systems in the context of the present invention are in particular carbonic acid/bicarbonate buffer, carbonic acid/silicate buffer, acetic acid/acetate buffer, phosphate buffer, ammonia buffer, citric acid or citrate buffer, buffer based on tris(hydroxymethyl)aminomethane, buffer based on 4-(2-hydroxyethyl)-1-piperazineethanesulfonic acid, buffer based on 4-(2-hydroxyethyl)piperazine-1-propanesulfonic acid, buffer based on 2-(N-morpholino)ethanesulfonic acid, and barbital/acetate buffer. The choice of suitable buffer system depends here on the desired pH of the antiperspirant cosmetics according to the invention.

In one preferred embodiment, the antiperspirant cosmetics according to the invention are characterized in that they include, based on the total weight of the antiperspirant cosmetic according to the invention,
at least one protein in a total amount of 0.5 to 60% by weight, preferably 1.0 to 50% by weight, preferably 1.5 to 40% by weight, more preferably 2.0 to 30% by weight, in particular 2.0 to 20% by weight,
12 to 98% by weight, preferably 25 to 55% by weight, preferably 30 to 50% by weight, in particular 35 to 45% by weight water,
at least one emulsifier and/or one surfactant,
at least one pH adjuster,
at least one preservative, and
at least one substance selected from the group consisting of cosmetic oils which are liquid at 20° C. and 1013 hPa, odorants and waxes,
   wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption.

In a further preferred embodiment, the antiperspirant cosmetics according to the invention are characterized in that they include, based on the total weight of the antiperspirant cosmetic according to the invention,
at least one protein in a total amount of 0.5 to 60% by weight, preferably 1.0 to 50% by weight, preferably 1.5 to 40% by weight, more preferably 2.0 to 30% by weight, in particular 2.0 to 20% by weight,
12 to 98% by weight, preferably 25 to 55% by weight, preferably 30 to 50% by weight, in particular 35 to 45% by weight water,
at least one emulsifier and/or one surfactant,
at least one pH adjuster,
at least one preservative,
0.01 to 2% by weight, preferably 0.1 to 1% by weight, preferably 0.2 to 0.7% by weight, in particular 0.3 to 0.5% by weight of a thickener, and
at least one substance selected from the group consisting of cosmetic oils which are liquid at 20° C. and 1013 hPa, odorants and waxes,
   wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption.

In one preferred embodiment, the antiperspirant cosmetics according to the invention are characterized in that they include, based on the total weight of the antiperspirant cosmetic according to the invention,
at least one protein in a total amount of 0.5 to 60% by weight, preferably 1.0 to 50% by weight, preferably 1.5 to 40% by weight, more preferably 2.0 to 30% by weight, in particular 2.0 to 20% by weight,
12 to 98% by weight, preferably 25 to 55% by weight, preferably 30 to 50% by weight, in particular 35 to 45% by weight water,
at least one propellant in a total amount of 1 to 98% by weight, preferably 20 to 90% by weight, preferably 30 to 85% by weight, in particular 40 to 75% by weight,
at least one emulsifier and/or one surfactant,
at least one pH adjuster,
at least one preservative, and
at least one substance selected from the group consisting of cosmetic oils which are liquid at 20° C. and 1013 hPa, odorants and waxes,
   wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption.

In a further preferred embodiment, the antiperspirant cosmetics according to the invention are characterized in that they include, based on the total weight of the antiperspirant cosmetic according to the invention,
at least one protein in a total amount of 0.5 to 60% by weight, preferably 1.0 to 50% by weight, preferably 1.5 to 40% by weight, more preferably 2.0 to 30% by weight, in particular 2.0 to 20% by weight,
12 to 98% by weight, preferably 25 to 55% by weight, preferably 30 to 50% by weight, in particular 35 to 45% by weight water,
at least one propellant in a total amount of 1 to 98% by weight, preferably 20 to 90% by weight, preferably 30 to 85% by weight, in particular 40 to 75% by weight,
at least one emulsifier and/or one surfactant,
at least one pH adjuster,
at least one preservative,
0.01 to 2% by weight, preferably 0.1 to 1% by weight, preferably 0.2 to 0.7% by weight, in particular 0.3 to 0.5% by weight of a thickener, and
at least one substance selected from the group consisting of cosmetic oils which are liquid at 20° C. and 1013 hPa, odorants and waxes,
   wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption.

Within the context of the present invention, it may also be provided to package the cosmetic according to the invention as a two-component cosmetic. The individual components are to this end preferably stored in separate containers and are applied to the skin in any order one after the other or simultaneously. A separation into multicomponent systems is particularly preferred where incompatibilities of the ingredients are to be expected or feared.

A further subject matter of the present invention is therefore a packaging unit (kit-of-parts) comprising, packaged separately from one another,
a) at least one first container (C1), including a cosmetic (M1) comprising at least one antiperspirant active substance, and
b) at least one second container (C2), including a cosmetic (M2) comprising at least one protein, wherein the at least one protein occurs in legumes of the genus Glycine, wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption, and wherein the cosmetic includes no aluminum and/or zirconium halides and/or hydroxy halides.

According to the invention, the term “antiperspirant active substance” will be understood to mean active substances which decrease or reduce the perspiration of the sweat glands of the body. However, the proteins from legumes of the genus Glycine which are included in the cosmetic (M2) and which bring about a change in light absorption under the conditions described above do not fall under this definition.

With regard to the cosmetic (M2) in the container (C2), what has been stated in relation to the cosmetics according to the invention applies mutatis mutandis.

A further subject matter of the present invention is the use of a protein for at least partially influencing the sweat gland(s), wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption.

According to the invention, influencing the sweat gland(s) will be understood to mean influencing the sweat gland(s) to the effect that the secretion of sweat from the efferent duct will be prevented or reduced. Without wishing to be limited to one theory, this may take place for example by forming a gel and/or a deposit of the at least one specific protein in the efferent duct of the sweat gland or in the efferent ducts of the sweat glands. However, the use of the at least one specific protein may also lead to a disruption of the charge equilibrium within the efferent ducts of the sweat glands. With regard to the use according to the invention, what has been stated in relation to the antiperspirant cosmetics according to the invention applies mutatis mutandis.

In addition, a further subject matter of the present invention is the use of a combination including
a) at least one substance selected from the group consisting of cosmetic oils which are liquid at 20° C. and 1013 hPa, odorants and waxes,
b) propellants in a total amount of 0 to 99% by weight, based on the total weight of the antiperspirant cosmetic, and
c) at least one protein in a total amount of 0.1 to 70% by weight, based on the total weight of the antiperspirant cosmetic, wherein the at least one protein occurs in legumes of the genus Glycine, wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption, and
   wherein the combination includes no aluminum and/or zirconium halides and/or hydroxy halides,
   to reduce and/or prevent perspiration, in particular underarm perspiration or perspiration from other body regions.

Within the context of the present invention, the term “combination” comprises a mixture of ingredients a), b) and c) specified above. With regard to the use of the aforementioned combination, what has been stated in relation to the antiperspirant cosmetics according to the invention and in relation to the use according to the invention applies mutatis mutandis.

Moreover, a further subject matter of the present invention is an antiperspirant cosmetic including
a) at least one substance selected from the group consisting of cosmetic oils which are liquid at 20° C. and 1013 hPa, odorants and waxes,
b) propellants in a total amount of 0 to 99% by weight, based on the total weight of the antiperspirant cosmetic, and
c) at least one soybean protein in a total amount of 0.1 to 70% by weight, based on the total weight of the antiperspirant cosmetic, wherein the soybean protein is hydrolyzed and/or wherein the soybean protein includes a lauryldimoniumhydroxypropyl group,
   wherein the antiperspirant cosmetic includes no aluminum and/or zirconium halides and/or hydroxy halides.

Within the context of this subject matter, it is particularly preferred if the at least one protein includes lysine in 1.5 to 6.5 mol %, histidine in 1.0 to 2.5 mol % and arginine in 4.5 to 6.5 mol %, based on the total amount of all amino acids of the protein. The content of the aforementioned amino acids can be determined in the manner explained above.

Furthermore, within the context of this subject matter, it is particularly preferred if the at least one protein has a calcium content of 10 to 600 mg, a potassium content of 650 to 850 mg, a phosphorus content of 800 to 1400 mg and a sulfur content of 1000 to 1600 mg, in each case based on 1 kg of the protein. These contents can be determined for example by means of atom emission spectrometry (ICP-OES), as explained above.

With regard to further preferred embodiments of this subject matter, what has been stated in relation to the antiperspirant cosmetics according to the invention, in relation to the use according to the invention and in relation to the method according to the invention applies mutatis mutandis.

Finally, a further subject matter of the present invention is a non-therapeutic cosmetic method for preventing and/or reducing body perspiration, in which an antiperspirant cosmetic according to the invention is applied to the skin, in particular to the skin of the underarms, and remains on the skin of the underarms for at least 1 hour, preferably for at least 2 hours, preferably for at least 4 hours, in particular for at least 6 hours.

However, within the context of the method according to the invention, it may also be provided that first a cosmetic including at least one antiperspirant aluminum and/or zirconium halide and/or hydroxy halide is applied, and then the cosmetic according to the invention is applied. However, it is also possible first to apply the cosmetic according to the invention and then to use a cosmetic including at least one antiperspirant aluminum and/or zirconium halide and/or hydroxy halide. The antiperspirant cosmetic according to the invention and the cosmetic including at least one antiperspirant aluminum and/or zirconium halide and/or hydroxy halide can also be applied to the skin simultaneously. The time interval between the application of the two cosmetics is from 0 seconds to 24 hours. It is also preferred if the cosmetics, after being applied, remain on the skin of the underarms for at least 1 hour, preferably for at least 2 hours, preferably for at least 4 hours, in particular for at least 6 hours.

With regard to the method according to the invention, what has been stated in relation to the antiperspirant cosmetics according to the invention and in relation to the use according to the invention applies mutatis mutandis.

The following examples explain the present invention, but without limiting the latter:

### Examples

1. Change in Light Absorption

The following proteins from legumes of the genus Glycine (soy):
1) Hydrolyzed soy protein having at least one lauryldimoniumhydroxypropyl group and an average molecular weight M_{w} of around 3000 Da and
2) Hydrolyzed soy protein having an average molecular weight M_{w} of around 2000 Da.

The change in light absorption brought about by the aforementioned proteins in a pH range from 4.0 to 8.0 in the event of a pH change of at least 0.5 is determined as follows:

TABLE 1
Sample solution (figures specified in % by weight)

| | EI* | E-II* |
|---|---|---|
| Hydrolyzed soy protein having at least one | 0.5 | — |
| lauryldimoniumhydroxypropyl group ^{a)} | | |
| Hydrolyzed soy protein ^{b)} | — | 5.7 |
| HCl | Add pH | Add pH |
| Water | Add 100 | Add 100 |

* according to the invention
^{a)} Croquat Soja PE-LQ (WD); (INCI: Lauryldimonium Hydroxypropyl Hydrolyzed Soy Protein; 20% strength by weight solution in water; M_{w}, around 3000 Da; Croda)
^{b)} Hydrosoy 2000 PE-LQ (WD); (INCI: Hydrolyzed Soy Protein; 17.5% strength by weight solution in water; M_{w}, around 2000 Da; Croda)

To determine the change in light absorption, use was made of a Methrom Titrando 905 from the company Methrom (USA), which is equipped with a Methrom Optrode 6.1115.000 and with a pH electrode from Methrom. The Methrom Titrando 905 is controlled via the Tiamo software from the company Methrom. First, 30 ml of a sample solution according to Table 1, which had a pH of 3.0, were placed in the open sample vessel of the Methrom Titrando 905. A 1% strength by weight sodium hydrogen carbonate solution was then continuously added at 23° C. and 1013 mbar, with stirring (stirring speed 8 of the Titrando 905, corresponding to approximately 750 to 850 rpm), until a pH of 7.5 was achieved. During the addition of the 1% strength by weight sodium hydrogen carbonate solution, the light transmission of a light beam through this sample solution was measured in mV (resolution 0.1 mV) using a Methrom Optrode 6.1115.000 at a wavelength of 574 nm (green-yellow). Each measurement is carried out twice, and the mean value is formed therefrom.

The change in light absorption brought about by the peptides mentioned above was determined according to the formula ΔL=[(|Lᵢ|/|L₀|]*100. In said formula, Lᵢ is the light transmission after a pH change of at least 0.5 in the pH range from 4.0 to 8.0, preferably from pH 4.5 and 7.5, in particular from pH 5.0 and 7.0. In this formula, L₀ is the difference of the light transmission at pH 4.0 and at pH 8.0, preferably at pH 4.5 and at pH 7.5, in particular at pH 5.0 and at pH 7.0.

With a pH change of 1.0 between pH 5.0 and pH 6.0 (light absorption at pH 6.0 forms value Lᵢ) in a pH range from 4.5 to 7.5 (difference in light absorption at pH 7.5 minus pH 4.5 forms value L₀), these proteins brought about the change in light absorption ΔL indicated in Table 2.

TABLE 2
Change in light absorption ΔL

| Sample solution | ΔL [%] |
|---|---|
| E-I | 61 |
| E-II | 31 |

2. In-Vivo Test of Antiperspirant Effect

In order to determine the antiperspirant effect, an antiperspirant study was carried out on the backs of 16 test subjects. The following antiperspirants were used for this:

| Antiperspirant | No. |
|---|---|
| Aqueous solution including 10% ACH, pH 4 | V-I |
| Aqueous solution including 5%* protein ^{a)}, | E-III** |
| pH 2-4 | |
| Aqueous solution including 5%* protein ^{b)}, | E-IV** |
| pH 2-4 | |

* active substance
** according to the invention
^{a)} Croquat Soja PE-LQ (WD); (INCI: Lauryldimonium Hydroxypropyl Hydrolyzed Soy Protein; 20% strength by weight solution in water; M_{w}, around 3000 Da; Croda)
^{b)} Hydrosoy 2000 PE-LQ (WD); (INCI: Hydrolyzed Soy Protein; 17.5% strength by weight solution in water; M_{w}, around 2000 Da; Croda)

On the backs of 16 test subjects, 40 μl of the antiperspirant V-I and 75 μl of the cosmetics according to the invention E-III and E-IV were in each case applied to one side next to the spine. After 5 minutes, the treated sites were covered with occlusive, non-adsorbent film. After 2 hours, these non-adsorbent pads were removed. The compositions were applied to the backs of the test subjects on four successive days, in each case in the aforementioned manner. 24 h after the last application of the composition, absorbent pads were applied to the backs of the test subjects at the sites where previously the compositions had been applied. Pads were also applied to the other side of the spine at the same height, these serving as control. After the test subjects had sweated for around 15 minutes at 80° C. in the sauna, the amount of sweat absorbed by the pads was determined gravimetrically, wherein each composition was compared with the respective corresponding untreated site on the back. From the gravimetric determination of the amount of sweat, the reduction in sweat was determined, wherein all the determined values were statistically significant.

The sweat reduction of the respective composition in comparison to an untreated skin site is shown in the following table:

| No. | Sweat reduction |
|---|---|
| V-I | 50% |
| E-III | 17% |
| E-IV | 13% |

The use of the specific protein leads to a significant decrease in sweat reduction and to a satisfactory antiperspirant effect.

3. Formulations:

The protein from legumes of the genus Glycine (soy) which is used in the following examples is preferably selected from the following proteins:
1) Hydrolyzed soy protein having at least one lauryldimoniumhydroxypropyl group and an average molecular weight M_{w} of around 3000 Da and
2) Hydrolyzed soy protein having an average molecular weight M_{w} of around 2000 Da.

Antiperspirant cosmetics according to the invention having a pH of 2.5 to 10.0 (figures specified in % by weight)

| | 1 | 2 | 3 | 4 | 5 | 6 | 7 |
|---|---|---|---|---|---|---|---|
| Isopropyl myristate | 0.50 | 0.10 | 0.50 | 1.0 | 2.0 | 3.0 | 5.0 |
| Protein | 0.50 | 2.0 | 3.0 | 5.0 | 7.0 | 10 | 20 |
| Eumulgin B3 ^{c)} | 3.0 | 3.0 | 3.0 | 4.0 | 4.0 | 4.0 | 5.0 |
| Perfume | 0.10 | 0.20 | 0.30 | 0.30 | 0.50 | 0.8 | 1.0 |
| Preservative | 0.50 | 0.50 | 0.50 | 0.80 | 0.80 | 1.5 | 2.0 |
| pH adjuster | ad pH | ad pH | ad pH | ad pH | ad pH | ad pH | ad pH |
| Water | ad 100 | ad 100 | ad 100 | ad 100 | ad 100 | ad 100 | ad 100 |

^{c)} Eumulgin B3 (INCI: Ceteareth-30; BASF)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic including
a) at least one substance selected from the group consisting of cosmetic oils which are liquid at 20° C. and 1013 hPa, odorants and waxes,
b) propellants in a total amount of 0 to 99% by weight, based on the total weight of the antiperspirant cosmetic, and
c) at least one protein in a total amount of 0.1 to 70% by weight, based on the total weight of the antiperspirant cosmetic, wherein the at least one protein occurs in legumes of the genus Glycine, and wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption,
wherein the antiperspirant cosmetic includes no aluminum and/or zirconium halides and/or hydroxy halides.

2. The antiperspirant cosmetic according to claim 1, wherein the at least one protein is included in a total amount of 0.5 to 60% by weight based on the total weight of the antiperspirant cosmetic.

3. The antiperspirant cosmetic according to claim 1, wherein the at least one protein is included in a total amount of 2 to 40% by weight based on the total weight of the antiperspirant cosmetic.

4. The antiperspirant cosmetic according to claim 1, wherein the at least one protein is included in a total amount of 3 to 20% by weight based on the total weight of the antiperspirant cosmetic.

5. The antiperspirant cosmetic according claim 1, wherein the at least one protein has a weight average molecular weight M_{w} of 150 to 100,000 Da.

6. The antiperspirant cosmetic according claim 1, wherein the at least one protein has a weight average molecular weight M_{w} of 180 to 50,000 Da.

7. The antiperspirant cosmetic according claim 1, wherein the at least one protein has a weight average molecular weight M_{w} of 200 to 10,000 Da.

8. The antiperspirant cosmetic according claim 1, wherein the at least one protein has a weight average molecular weight M_{w} of 250 to 8000 Da.

9. The antiperspirant cosmetic according claim 1, wherein the at least one protein has a weight average molecular weight M_{w} of 300 to 5000 Da.

10. The antiperspirant cosmetic according to claim 1, wherein the at least one protein brings about a change in light absorption in the event of a pH change of at least 0.5 in a pH range from pH 4.5 to pH 7.5 at a concentration of 0.001 to 10% by weight protein, based on the total weight of the sample mixture used for the pH measurement, and at a temperature of 20° C.

11. The antiperspirant cosmetic according to claim 1, wherein the at least one protein brings about a change in light absorption in the event of a pH change of at least 0.5 in a pH range from pH 5.0 to pH 7.0 at a concentration of 0.001 to 10% by weight protein, based on the total weight of the sample mixture used for the pH measurement, and at a temperature of 20° C.

12. The antiperspirant cosmetic according to claim 1, wherein the pH change takes place by adding hydrogen carbonates or carbonates.

13. The antiperspirant cosmetic according to claim 1, wherein the at least one protein is selected from the group consisting of (i) unmodified proteins; (ii) hydrolyzed proteins; (iii) chemically modified proteins; (iv) physically modified proteins; (v) hydrolyzed unmodified proteins; (vi) hydrolyzed and chemically modified proteins;
(vii) hydrolyzed and physically modified proteins; and (viii) mixtures thereof.

14. The antiperspirant cosmetic according to claim 1, wherein the at least one protein is a cationically modified protein having one or more radical(s) of formula R¹—N⁺(CH₃)₂—CH₂—CH(OH)—CH₂—X—R, in which R¹ represents an alkyl group having 1 to 30 carbon atoms, an alkenyl group having 1 to 30 carbon atoms, a hydroxyalkyl group having 1 to 30 carbon atoms, a C₁₀₋₁₈ alkyl or a C₁₀₋₁₈ alkenyl group, X represents O, N or S, and R represents the protein radical.

15. The antiperspirant cosmetic according to claim 1, wherein the at least one protein is hydrolyzed and/or the at least one protein includes at least one lauryldimoniumhydroxypropyl group.

16. The antiperspirant cosmetic according to claim 1, wherein the at least one protein includes lysine in 1.5 to 6.5 mol %, histidine in 1.0 to 2.5 mol % and arginine in 4.5 to 6.5 mol %, based on the total amount of all amino acids of the protein.

17. The antiperspirant cosmetic according to claim 16, wherein the at least one protein has a calcium content of at least 10 to 600 mg, a potassium content of 650 to 850 mg, a phosphorus content of 800 to 1400 mg and a sulfur content of 1000 to 1600 mg, in each case based on 1 kg of the protein.

18. A non-therapeutic cosmetic method for preventing and/or reducing body perspiration, wherein the antiperspirant cosmetic according to claim 1 is applied to the skin, and left on the skin of the underarms for at least 1 hour.

19. A packaging unit (kit-of-parts) comprising, packaged separately from one another,
a) at least one first container (C1), including a cosmetic (M1) comprising at least one antiperspirant active substance, and
b) at least one second container (C2), including a cosmetic (M2) comprising at least one protein, wherein the at least one protein occurs in legumes of the genus Glycine, wherein the at least one protein brings about a change in light absorption of 1 to 100% in the event of a pH change of at least 0.5 in a pH range from pH 4.0 to pH 8.0, at a temperature of 20° C. to 40° C. and at a concentration of the protein of 0.001 to 10% by weight, based on the total weight of the sample mixture used to determine the change in light absorption, and wherein the cosmetic (M2) includes no aluminum and/or zirconium halides and/or hydroxy halides.

20. An antiperspirant cosmetic, comprising:
a) at least one substance selected from the group consisting of cosmetic oils which are liquid at 20° C. and 1013 hPa, odorants and waxes,
b) propellants in a total amount of 0 to 99% by weight, based on the total weight of the antiperspirant cosmetic, and
c) at least one soybean protein in a total amount of 0.1 to 70% by weight, based on the total weight of the antiperspirant cosmetic, wherein the soybean protein is hydrolyzed and/or wherein the soybean protein includes a lauryldimoniumhydroxypropyl group,
wherein the antiperspirant cosmetic includes no aluminum and/or zirconium halides and/or hydroxy halides.
